(19) United States
(12) Patent Application Publication
(10) Pub. No.: US 2006/0089606 A1
(43) Pub. Date: Apr. 27, 2006

(54) Medicine-adding port of vein infusion set and catheter needle set

(76) Inventors: Ming Hsiung Tsai, Taichung City (TW); Chia Han Tsai, Taichung City (TW); Tsai Chia Mu, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/971,036

(22) Filed: Oct. 25, 2004

(51) Int. Cl. A61M 5/14

(52) U.S. Cl. 604/256

(57) ABSTRACT

A drug administration port of infusion set and catheter needle set, including: a drug administration socket having a drug through hole, a free end of the drug administration socket being formed with a seat defining an opening communicating with the drug through hole; a plastic plug for blocking the opening of the seat; and a connecting member connected with the free end of the drug administration socket. The connecting member has a tapered hole axially positioned on the upper face of the plastic plug. A small diameter end of the tapered hole faces the plastic plug. A threaded section is formed on an opposing end of the connecting member. An upper face of the plastic plug is cut with a split axially corresponding to the position of the tapered hole. The split serves to communicate with the drug through hole.

### BACKGROUND OF THE INVENTION

The present invention is related to a drug administration port of an infusion set and catheter needle set. Both the injection syringe equipped with a needle or without a needle can be inserted into the drug administration port for adding the drug.

FIGS. 12 and 13 show a conventional infusion set including a plastic needle 71 inserted in an infusion bag 72 or drug bottle. The plastic needle 71 via a flexible tube 73 is connected to a volumetric burette 74. A flow controller (not shown) and a drug administration tube 78 are arranged between the burette 74 and the injection needle 75. When it is necessary to give a drug to a patient, the drug can be selectively filled into the drug administration port 762 of the burette 74 or directly injected into the drug administration port 761 of the drug administration tube 78, depending on the usage of the drug. Alternatively, the drug can be added through the drug administration port 763 of the infusion bag. In some cases, the drug is directly injected into the vessel of the patient from a drug administration port of a catheter needle. During such procedure, a nurse withdraws the drug into a syringe and then thrusts the injection needle 78 into one of the drug administration ports. The above drug administration ports have some shortcomings in the actual drug administration operation. For example, each of the drug administration ports 761, 762, 763 has a rubber plug 77. The injection needle must be first be thrust through the rubber plug 77 and then the drug can be injected. Medical workers are often incautiously impaled by the injection needle when thrusting the injection needle into the rubber plug as shown in FIG. 14. This is because the drug administration port has a too small a size and difficult to hold. Also, the rubber plug 77 has a quite small area. When a nurse holds the syringe 75 with one hand, it is hard to aim the injection needle at the rubber plug 77 and precisely thrust the needle into the rubber plug. As a result, impalement often takes place. Especially in clinical settings, it is more inconvenient for a medical worker to thrust the injection needle into the drug administration port 762 of the burette 74 or the drug administration port 763 of the infusion bag 72 which is placed in a relatively high position. Under such circumstance, the medical worker is more apt to become impaled.

### SUMMARY OF THE INVENTION

It is therefore a primary object of the present invention to provide a drug administration port of an infusion set and catheter needle set. The drug administration port can be conveniently and more safely used.

It is a further object of the present invention to provide the above drug administration port of an infusion set and catheter needle set. Both the injection syringe equipped with a needle or without a needle can be inserted into the drug administration port for adding the drug.

According to the above objects, the drug administration port of an infusion set and catheter needle set includes: a drug administration socket having a drug through hole, a free end of the drug administration socket being formed with a seat defining an opening communicating with the drug through hole; a plastic plug for blocking the opening of the seat; and a connecting member connected with the free end of the drug administration socket. The connecting member has a tapered hole axially positioned on the upper face of the plastic plug. A small diameter end of the tapered hole faces the plastic plug. A threaded section is formed on a large diameter end of the tapered hole. An upper face of the plastic plug is cut with a split axially corresponding to the position of the tapered hole. The split serves to communicate with the drug through hole.

The present invention can be best understood through the following description and accompanying drawings wherein:

### BRIEF DESCRIPTION OF THE DRAWINGS

FIG. 1 shows that the drug administration port of the present invention is disposed on an infusion set;

FIG. 2 is a perspective assembled view showing that the drug administration port of the present invention is disposed on a flexible tube of an infusion set;

FIG. 3 is a perspective exploded view of the drug administration tube and drug administration port of FIG. 1;

FIG. 4 is a partially sectioned assembled view showing that the drug administration port of the present invention is disposed on the drug administration tube;

FIG. 5 is a view according to FIG. 4, showing that the needle holder of a syringe being directly inserted into the tapered hole of the drug administration port of the present invention;

FIG. 6 is an illustration of a syringe equipped with a plastic safety needle i- inserted into the drug administration port of the present invention;

FIG. 7 is an illustration of a lock-type syringe is inserted into the drug administration port of the present invention;

FIG. 8 is an illustration of the drug administration port of the present invention disposed on an upper side of the volumetric burette of the infusion set;

FIG. 9 is an illustration of the drug administration port of the present invention disposed on the infusion bag of the infusion set;

FIG. 10 is partially sectioned view showing the drug administration port of the present invention is disposed on a catheter needle;

FIG. 11 is an illustration of the catheter needle with the drug administration port applied to a patient's wrist;

FIG. 12 is an illustration of a conventional infusion set;

FIG. 13 is a perspective view of the drug administration tube of the conventional infusion set; and

FIG. 14 is an illustration of a medical worker inserting an injection needle into the drug administration port of the drug administration tube and inadvertently becoming impaled.

### DETAILED DESCRIPTION OF THE PREFERRED EMBODIMENTS

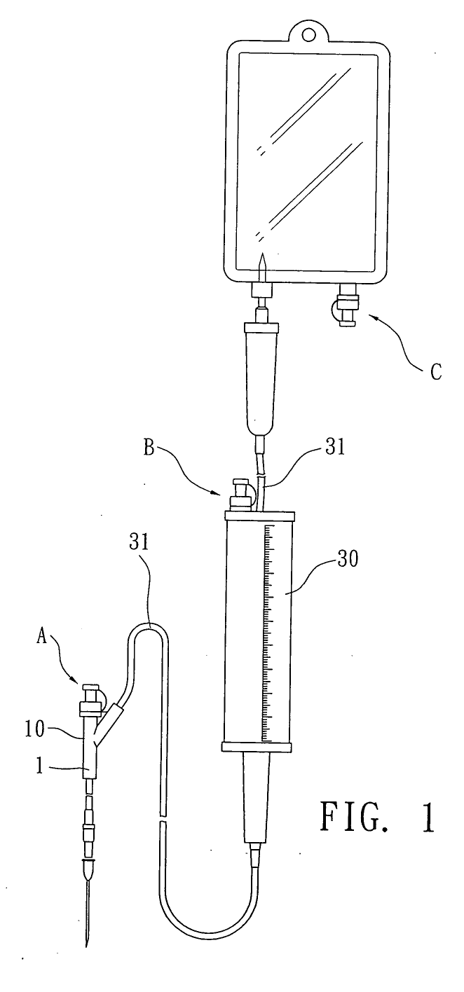

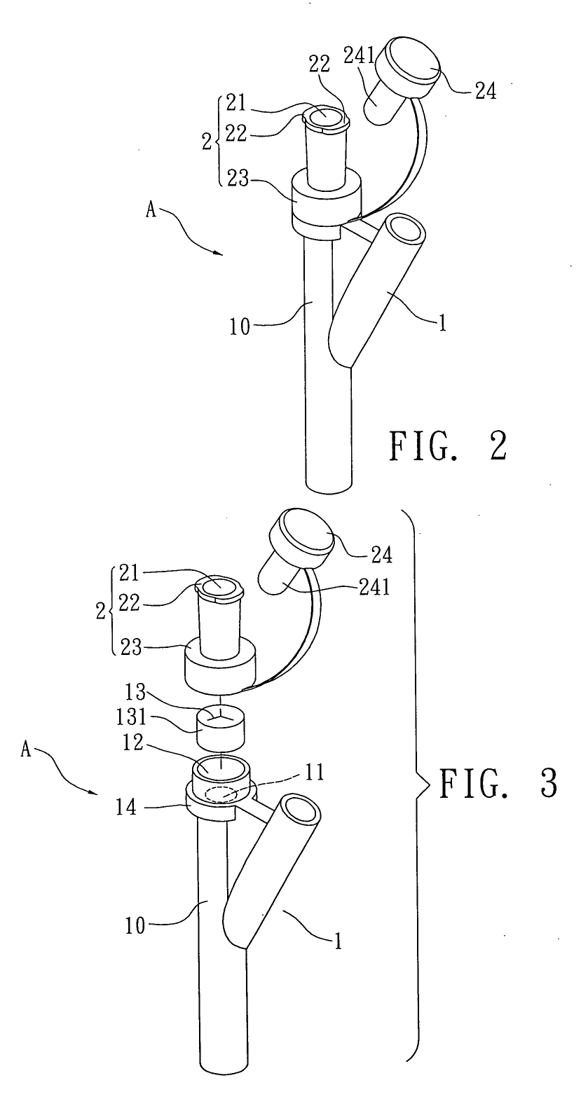

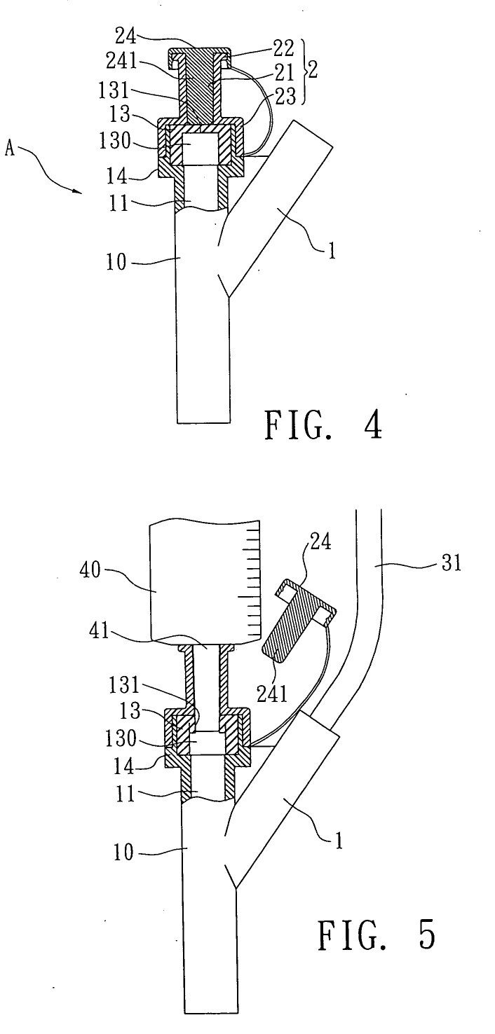

Please refer to FIG. 1 which shows an infusion set. Also referring to FIGS. 2 to 4, the drug administration port A is disposed on the drug administration tube 1 of the flexible tube 31 of the infusion set. The drug administration port A includes a drug administration socket 10 having a drug through hole 11 for communicating with the drug administration tube 1. A free end of the drug administration socket 10 is formed with a seat 12 defining an enlarged opening communicating with the drug through hole 11. A plastic plug 13 blocks the seat 12. A lower face of the plastic plug 13 is formed with an axial deep blind hole 130. An upper face of the plastic plug is cut with a split 131 within a range of the diameter of the blind hole. The split 131 serves to communicate with the drug through hole 11. The split can be Y-shaped, +-shaped, V-shaped, L-shaped or I-shaped.

A connecting member 2 is connected with the free end of the drug administration socket 10. The connecting member 2 has a tapered hole 21 axially positioned on the upper face of the plastic plug 13. The small diameter end of the tapered hole 21 faces the plastic plug 13. The connecting member 2 further has a cap 23 formed at the small diameter end of the tapered hole 21. The cap 23 is fitted around the seat 12 of the free end of the drug administration socket 10. Two outer flanges 22 are formed on the large diameter end of the tapered hole 21 of the connecting member 2. The outer flanges 22 form a threaded section. A protective plug 24 is fitted on the connecting member 2 for blocking the large diameter end of the tapered hole 21.

The plastic plug 13 can be tightly fitted in the seat 12 of the drug administration socket 10. The cap 23 of the connecting member 2 has an inner diameter and depth such that the cap 23 can be snugly fit on the seat 12 to press the plastic plug 13 without detachment. A circumference of the cap 23 abuts against a stop section 14 formed around the seat 12, whereby the cap 23 can entirely cover the seat 12. The connecting member 2 is tightly connected with the seat 12 by means of an adhesive or high-frequency wave bonding.

According to the above structure, as shown in FIG. 5, a common syringe 40 without an injection needle can be inserted into the drug administration port of the present invention for adding the drug. The needle holder 41 at front end of the syringe is inserted into the tapered hole 21 to press the split 131 formed on the upper face of the plastic plug 13. There after, the needle holder 41 thrusts through the split 131 to form an opening in which the needle holder 41 is held. At this time, the needle holder 41 is tightly fit in the tapered hole 21 and stably held. Accordingly, a medical worker can add the drug from the syringe 40 into the drug administration socket 10.

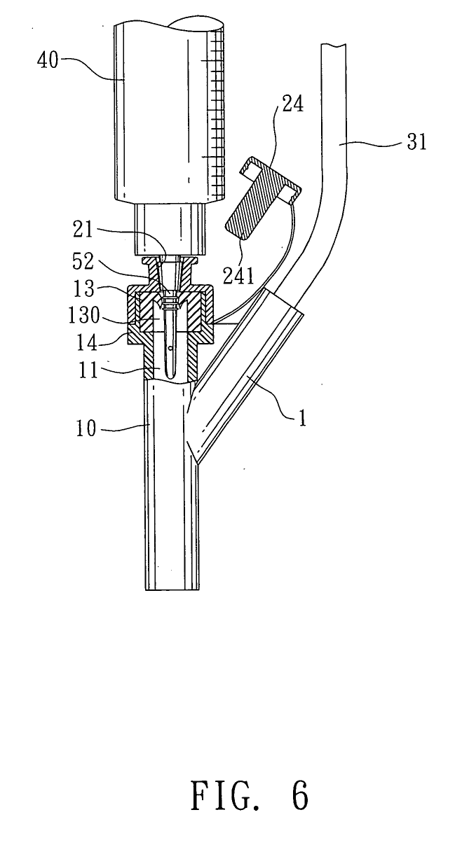

FIG. 6 shows that the syringe 40 is equipped with a plastic safety needle 52 which cooperates with the drug administration port of the present invention to add the drug into the infusion set.

According to the above embodiment, both the plastic safety needle 52 and the needle holder 41 are larger than a common metal needle. However, the axially extending wall of the plastic plug, which defines the blind hole 130 is able to stably locate the plastic plug. Therefore, in actual application, when the plastic safety needle 52 or the needle holder 41 is thrust through the plastic plug, the plastic plug will not be pushed into the drug administration socket 10. On the other hand, after the injection is completed, when extracting the syringe, although the petals of the split 131 of the plastic plug 13 still tightly clamp the needle holder 41 or the plastic safety needle 52, the cap 23 can stop the plastic plug 13 from being extracted therewith.

The present invention is characterized in that all types of needles and the needle holder 41 (as shown in FIGS. 5, 6 and 7) can be directly safely and stably inserted into or extracted out of the drug administration port. This is because the plastic plug 13 is precisely adapted to the seat 12 of the drug administration socket 10. The plastic plug 13 is cut with the split 131. When the needle holder 41 presses the plastic plug 13, the split 131 will be forcedly stretched open to form a flow passage. Accordingly, the drug can be filled into the drug administration port.

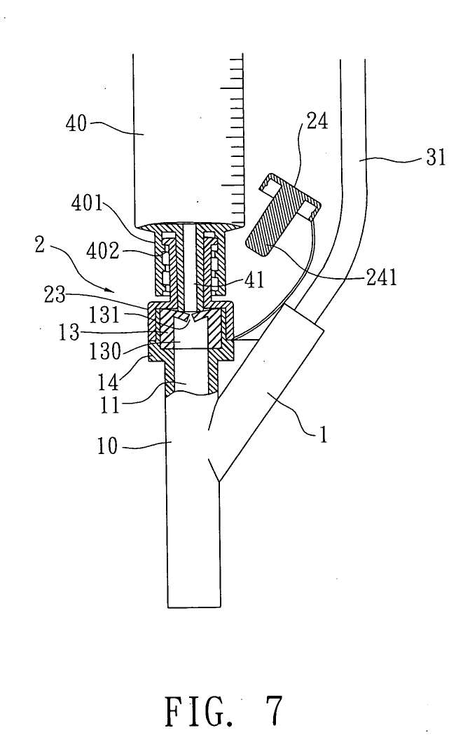

It should be noted that the outer flanges 22 of the present invention is co-usable with a Luer lock syringe 40 as shown in FIG. 7. The needle holder 41 of the syringe 40 is formed with a threaded section 401 having inner thread 402. The threaded section of the syringe 40 can be screwed onto the outer flanges 22. At the same time, the needle holder 41 will press the split 131 of the upper face of the plastic plug 13 to form an opening. By means of the threaded engagement, the syringe can be more accurately and firmly associated with the drug administration port. In addition, the outer flanges 22 can fix the luer lock syringe so that the medical worker does not need to hold and push the syringe with both hands. Accordingly, the drug administration operation can be easily performed.

Moreover, after the drug is added, the protective plug 24 can be tightly plugged into the tapered hole 21 of the connecting member. An inner stem 241 of the protective cover can abut against the plastic plug 13 to prevent the drug from leaking out.

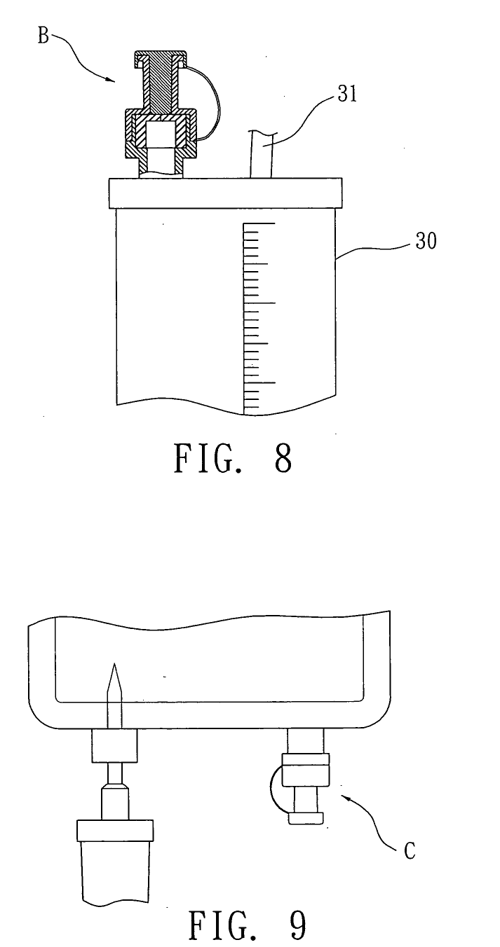

FIG. 8 shows a second embodiment of the present invention, in which the drug administration port is disposed in position B on upper side of the volumetric burette 30 of the infusion set. FIG. 9 shows that the drug administration port is disposed in position C under the infusion bag of the infusion set. These embodiments can achieve the same function as the first embodiment to facilitate the drug administration operation.

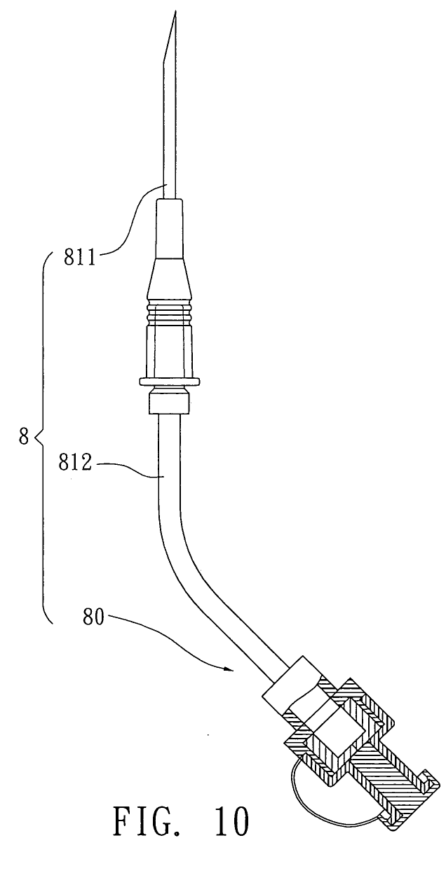

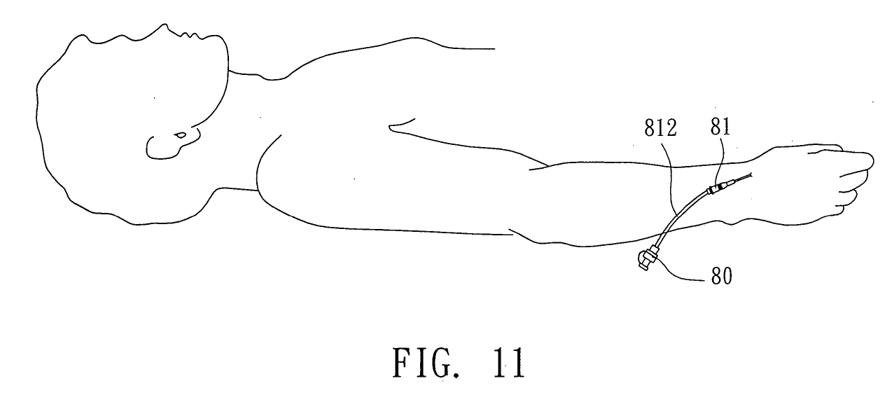

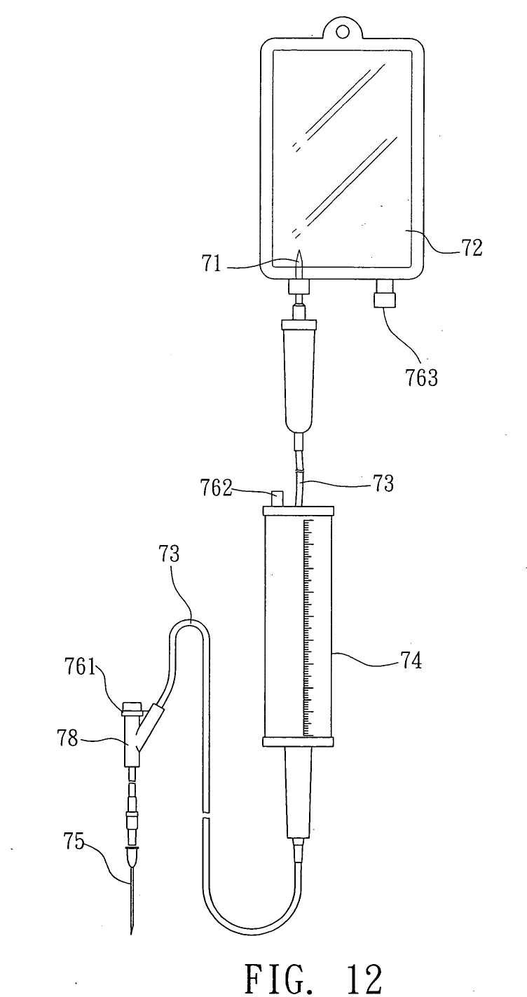

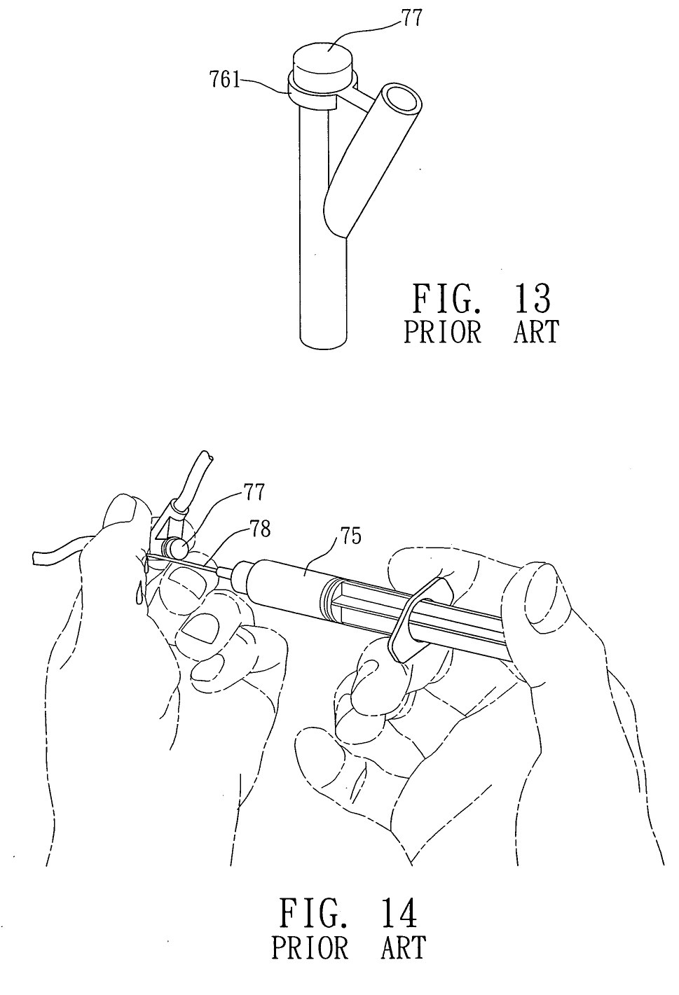

FIG. 10 shows a vein catheter needle set 8. The drug administration port 80 of the present invention is disposed at a rear end of a flexible tube 812 of a catheter needle 811. Referring to FIG. 11, the catheter needle 811 is inserted in a patient's wrist and the drug can be safely injected into the flexible tube 812 through the drug administration port 80.

The above embodiments are only used to illustrate the present invention, and are not intended to limit the scope thereof. Many modifications of the above embodiments can be made without departing from the spirit of the present invention.

1. A drug administration port of an infusion set and catheter needle set, comprising:
a drug administration socket having a drug through hole, the drug administration socket having a free end formed with a seat having an opening communicating with the drug through hole;
a plastic plug for blocking the opening of the seat; and
a connecting member connected with the free end of the drug administration socket, the connecting member having a tapered hole axially positioned on an upper face of the plastic plug, the tapered hole having a small diameter end facing the plastic plug and an opposing end of the connecting member having a threaded section formed thereon, the upper face of the plastic plug being cut with a split disposed in axially aligned relationship with the tapered hole, the split serving to communicate with the drug through hole.

2. The drug administration port of an infusion set and catheter needle set as claimed in claim 1, wherein the split of the upper face of the plastic plug has a contour selected from the group consisting of a Y-shape, a cross-shape, a V-shape, an L-shape and an I-shape.

3. The drug administration port of an infusion set and catheter needle set as claimed in claim 2, wherein a lower face of the plastic plug is formed with an axial blind hole, the split being formed on the upper face of the plastic plug within a range of a diameter of the blind hole.

4. The drug administration port of an infusion set and catheter needle set as claimed in claim 3, wherein the seat of the drug administration socket defines an opening with an enlarged diameter, the plastic plug being plugged in the seat to block the opening.

5. The drug administration port of an infusion set and catheter needle set as claimed in claim 4, wherein a cap is formed at the small diameter end of the tapered hole of the connecting member, the cap being fit on the free end of the drug administration socket.

6. The drug administration port of an infusion set and catheter needle set as claimed in claim 5, wherein a flange is formed on outer circumference of the drug administration socket to form a stop section against which a bottom end of the cap abuts.

7. The drug administration port of infusion set and catheter needle set as claimed in claim 6, wherein the threaded section is formed by two outer flanges extending from the connecting member.

8. The drug administration port of an infusion set and catheter needle set as claimed in claim 7, further comprising a protective plug tightly disposed in the tapered hole of the connecting member, the protective plug having a stem projecting from inner face thereof for abutting against the plastic plug.

9. (canceled)

10. The drug administration port of an infusion set and catheter needle set as claimed in claim 8, wherein the drug administration port is disposed on upper side of a volumetric burette of the infusion set.

11. The drug administration port of an infusion set and catheter needle set as claimed in claim 8, wherein the drug administration port is disposed on a drug administration tube of the flexible tube of the infusion set.

12. The drug administration port of an infusion set and catheter needle set as claimed in claim 8, wherein the drug administration port is disposed on an infusion bag of the infusion set.

13. The drug administration port of an infusion set and catheter needle set as claimed in claim 8, wherein the catheter needle set includes a flexible tube and a catheter needle disposed at a distal end of the flexible tube, the drug administration port being disposed at a proximal end of the flexible tube.
